(19) United States
(12) Patent Application Publication
(10) Pub. No.: US 2024/0076244 A1
(43) Pub. Date: Mar. 7, 2024

(54) METHOD OF PREDICTING CONCRETE CHARACTERISTICS

(71) Applicant: SAMSUNG ELECTRONICS CO., LTD., Suwon-si (KR)

(72) Inventors: Hyeongmin SON, Suwon-si (KR); Solmoi Park, Busan (KR); Aron Berhanu Degefa, Busan (KR); Hyejin Yu, Busan (KR); Hyekyung Youn, Suwon-si (KR)

(73) Assignees: SAMSUNG ELECTRONICS CO., LTD., Suwon-si (KR); Pukyong National University Industry-University Cooperation Foundation, Busan (KR)

(21) Appl. No.: 17/983,152

(22) Filed: Nov. 8, 2022

(30) Foreign Application Priority Data
Sep. 7, 2022 (KR) 10-2022-0113786

(51) Int. Cl. C04B 40/00; C04B 14/10; C04B 18/08; C04B 18/14; C04B 28/04; G06N 5/02

(52) U.S. Cl. CPC C04B 40/0032; C04B 14/106; C04B 18/08; C04B 18/141; C04B 18/146; C04B 28/04; G06N 5/022

(57) ABSTRACT

A method of predicting characteristics of concrete includes inputting data on a cement mixture including cement and an admixture, calculating reactivity of the admixture based on the data, and predicting products of the cement mixture based on the reactivity, and an embodiment further includes predicting characteristics of concrete based on the predicting of the products of the cement mixture based on the reactivity.

### CROSS-REFERENCE TO RELATED APPLICATION

This application is based on and claims priority to Korean Patent Application No. 10-2022-0113786, filed on Sep. 7, 2022, in the Korean Intellectual Property Office, the disclosure of which is incorporated by reference herein in its entirety.

### BACKGROUND

The present disclosure relates to a method of predicting concrete characteristics, and more particularly, to a method of predicting concrete characteristics including cement and an admixture.

Concrete is the most representative and widely used construction material, accounting for 80% of the world's structures. The components of concrete include cement, water, aggregates, and additives. Among the above components, cement accounts for the largest portion of the carbon dioxide generation of concrete.

Most concrete is manufactured using Portland cement as a binder. However, research is being conducted to reduce carbon generation of the binder as the amount of carbon dioxide of Portland cement matters.

### SUMMARY

The present disclosure relates to a method of predicting concrete characteristics in accordance with the chemical composition and weight ratio wt % of an admixture.

An object to be achieved by the present disclosure is not limited thereto and other objects that are not mentioned may be clearly understood by a person skilled in the art from the following description.

According to an aspect of the present disclosure, there is provided a method of predicting concrete characteristics, including inputting data on a cement mixture including cement and an admixture, calculating reactivity of the admixture based on the data, and predicting products of the cement mixture based on the reactivity.

According to another aspect of the present disclosure, there is provided a method of predicting concrete characteristics, including inputting data on a cement mixture including cement and an admixture, calculating reactivity of the admixture based on the data, predicting products of the cement mixture based on the reactivity, and predicting characteristics of concrete including the products based on the predicting of the products of the cement mixture based on the reactivity. The predicting of the concrete characteristics includes predicting an amount of net carbon dioxide generated in processes of manufacturing concrete from the cement mixture. The amount of net carbon dioxide means excluding an amount of carbon dioxide absorbed in the manufacturing processes from an amount of carbon dioxide generated in the manufacturing processes.

According to another aspect of the present disclosure, there is provided a method of predicting concrete characteristics, including inputting data on a cement mixture including cement and an admixture, calculating reactivity of the admixture based on the data, predicting products of the cement mixture based on the reactivity, predicting an amount of an air gap in concrete including the products based on the predicting of the products of the cement mixture based on the reactivity, and predicting compressive strength of concrete based on the predicting of the amount of the air gap in the concrete.

### BRIEF DESCRIPTION OF THE DRAWINGS

Embodiments will be more clearly understood from the following detailed description taken in conjunction with the accompanying drawings in which:

FIG. 1 is a flowchart illustrating a method of predicting concrete characteristics according to embodiments;

FIG. 2 is a flowchart illustrating another method of predicting concrete characteristics according to embodiments;

FIGS. 3A to 3D are graphs each illustrating a result of predicting products of cement mixtures by a method of predicting concrete characteristics according to embodiments;

FIGS. 4A to 4D are graphs each illustrating a result of predicting an amount of an air gap in concrete by a method of predicting concrete characteristics according to embodiments;

FIGS. 5A to 5D are graphs each illustrating a result of predicting compressive strength of concrete by a method of predicting concrete characteristics according to embodiments; and

FIGS. 6A to 6D are graphs each illustrating a result of predicting an amount of net carbon dioxide generated in manufacturing processes of concrete by a method of predicting concrete characteristics according to embodiments.

### DETAILED DESCRIPTION OF THE EMBODIMENTS

Hereinafter, embodiments of the present disclosure will be described in detail with reference to the accompanying drawings. Like reference numerals refer to like elements throughout and a description thereof will not be given. Hereinafter, thicknesses or sizes of the respective layers are exaggerated for convenience sake and may be different from real shapes and ratios.

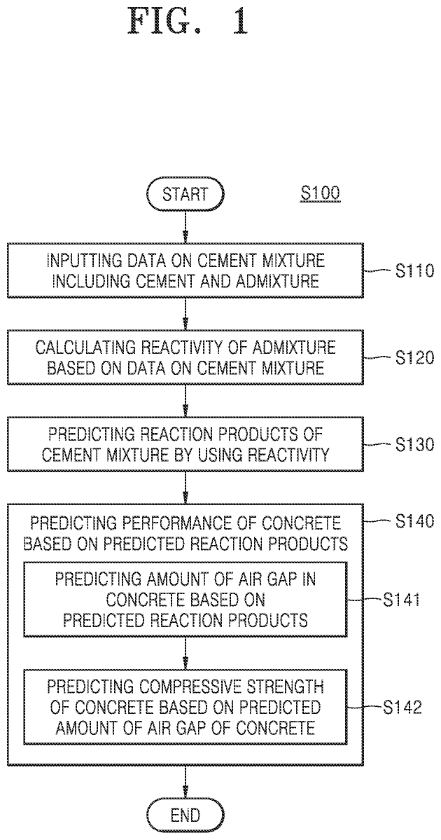

FIG. 1 is a flowchart illustrating a method S100 of predicting concrete characteristics according to embodiments.

According to the present specification, the concrete characteristics may include an amount of an air gap in concrete, the compressive strength of concrete, an amount of carbon dioxide generated in manufacturing processes of concrete, and performance of concrete.

Referring to FIG. 1, data of a cement mixture including cement and an admixture may be input in operation S110.

In some embodiments, cement may include Portland cement. In some embodiments, the admixture may include at least one of slag, fly ash, metakaolin, and silica fume. As the above-described admixture is mixed with Portland cement, the amount of Portland cement used and the amount of carbon dioxide emissions may be reduced. Although it is illustrated in the present specification that slag, fly ash, metakaolin, or silica fume is used as the admixture, the present disclosure is not limited thereto. A solid material that may have a characteristic value of the admixture such as a chemical composition or a specific surface area may be used as the admixture.

In some embodiments, the data may include a mixing ratio of the cement mixture, the chemical composition of cement and the chemical composition of the admixture, the specific surface area of the admixture, a curing condition of concrete, and the curing time of concrete.

Specifically, the data may include the ratio of each of the admixtures to cement in the cement mixture. For example, when the cement mixture includes Portland cement and slag, the data may include the ratio in which Portland cement and slag are mixed with each other. For example, when the cement mixture includes Portland cement and fly ash, the data may include the ratio in which Portland cement and fly ash are mixed with each other. For example, when the cement mixture includes Portland cement and metakaolin, the data may include the ratio in which Portland cement and metakaolin are mixed with each other. For example, when the cement mixture includes Portland cement and silica fume, the data may include the ratio in which Portland cement and silica fume are mixed with each other.

Specifically, cement and/or the admixture may include SiO₂, Al₂O₃, Fe₂O₃, CaO, MgO, SO₃, Na₂O, and/or K₂O, and the data may include the ratio in which the material is included in cement and/or the admixture.

Kinds of cement and/or admixtures used in experimental examples of embodiments and chemical compositions of the materials are illustrated in the following Table 1.

TABLE 1

| wt % | SiO₂ | Al₂O₃ | Fe₂O₃ | CaO | MgO | SO₃ | Na₂O | K₂O |
|---|---|---|---|---|---|---|---|---|
| Portland cement | 20.2 | 5.6 | 2.4 | 65.9 | 2.0 | 2.6 | 0.5 | 0.7 |
| (PC) | | | | | | | | |
| Slag | 36.49 | 12.26 | 0 | 41.79 | 7.48 | 1.98 | 0 | 0 |
| Fly ash | 51.8 | 23.4 | 7.2 | 10.8 | 2.7 | 1.1 | 1.3 | 1.6 |
| Metakaolin | 54.1 | 43.6 | 1.1 | 0.2 | 0.2 | 0.1 | 0.1 | 0.5 |
| Silica fume | 99.3 | 0.1 | 0 | 0.1 | 0.1 | 0 | 0.1 | 0.2 |

Referring to FIG. 1, reactivity of the admixture may be calculated in operation S120 based on the data input in operation S110.

In some embodiments, the reactivity may be calculated through the following Equation (1).

reactivity(wt %)=47.19×A−0.01144×G+0.1144×I−0.6373×M+0.00185×J×(M+D×H+H×K)+0.6373×\sqrt{C}+13.16×\sqrt{V}M+E²×(G−I)×(4×G+H)×0.0001544+0.01144×B×D×K×(F+G+L)−9.762 [EQUATION (1)]

The unit of reactivity calculated through Equation (1) is a weight ratio wt %. That is, a weight ratio wt % of a reactive admixture to a weight of an added admixture may be calculated through Equation (1). In Equation (1), A represents the ratio of the weight of water to the weight of the admixture. B represents the curing temperature ° C. C represents curing time, and the unit of the time is days. D represents the weight ratio wt % of SiO₂ included in cement to the gross weight of the cement, and the cement may include, for example, Portland cement. E represents the weight ratio wt % of CaO included in cement to the gross weight of the cement. F represents the weight ratio wt % of MgO included in cement to the gross weight of the cement. G represents the weight ratio wt % of SO₃ included in cement to the gross weight of the cement. H represents the weight ratio wt % of Al₂O₃ included in the admixture to the gross weight of the admixture. I represents the weight ratio wt % of Fe₂O₃ included in the admixture to the gross weight of the admixture. J represents the weight ratio wt % of CaO included in the admixture to the gross weight of the admixture. K represents the weight ratio wt % of SO₃ included in the admixture to the gross weight of the admixture. L represents the weight ratio wt % of Na₂O included in the admixture to the gross weight of the admixture. M represents the specific surface area of the admixture, and the unit of the specific surface area is m²/gm.

For example, referring to Table 1, when the cement mixture includes Portland cement and slag, the reactivity may be calculated in operation S120 based on the chemical composition of Portland cement and the chemical composition of slag. In other words, by substituting the chemical composition of Portland cement into Equation (1), that is, by substituting 20.2 for D, 65.9 for E, 2.0 for F, and 2.6 for G, by substituting the chemical composition of slag into Equation (1), that is, by substituting 12.26 for H, 0 for I, 41.79 for J, 1.98 for K, and 0 for L, and by inputting remaining values A, B, C, and M, the reactivity of the admixture, that is, slag, may be predicted.

For example, referring to Table 1, when the cement mixture includes Portland cement and fly ash, the reactivity may be calculated in operation S120 based on the chemical composition of Portland cement and the chemical composition of fly ash. In other words, by substituting the chemical composition of Portland cement into Equation (1), that is, by substituting 20.2 for D, 65.9 for E, 2.0 for F, and 2.6 for G, by substituting the chemical composition of fly ash into Equation (1), that is, by substituting 23.4 for H, 7.2 for I, 10.8 for J, 1.1 for K, and 1.3 for L, and by inputting the remaining values A, B, C, and M, the reactivity of the admixture, that is, fly ash, may be predicted.

For example, referring to Table 1, when the cement mixture includes Portland cement and metakaolin, the reactivity may be calculated in operation S120 based on the chemical composition of Portland cement and the chemical composition of metakaolin. In other words, by substituting the chemical composition of Portland cement into Equation (1), that is, by substituting 20.2 for D, 65.9 for E, 2.0 for F, and 2.6 for G, by substituting the chemical composition of metakaolin into Equation (1), that is, by substituting 43.6 for H, 1.1 for I, 0.2 for J, 0.1 for K, and 0.1 for L, and by inputting the remaining values A, B, C, and M, the reactivity of the admixture, that is, metakaolin, may be predicted.

For example, referring to Table 1, when the cement mixture includes Portland cement and silica fume, the reactivity may be calculated in operation S120 based on the chemical composition of Portland cement and the chemical composition of silica fume. In other words, by substituting the chemical composition of Portland cement into Equation (1), that is, by substituting 20.2 for D, 65.9 for E, 2.0 for F, and 2.6 for G, by substituting the chemical composition of silica fume into Equation (1), that is, by substituting 0.1 for H, 0 for I, 0.1 for J, 0 for K, and 0.1 for L, and by inputting the remaining values A, B, C, and M, the reactivity of the admixture, that is, silica, fume may be predicted.

In the present specification, slag, fly ash, metakaolin, and silica fume are used as the admixtures in the experimental examples. However, Equation (1) for calculating the reactivity may also be applied to other types of admixtures than the above-described admixtures. In other words, the present disclosure is not limited to using slag, fly ash, metakaolin, and/or silica fume as the admixtures/admixture.

Referring to FIG. 1, products of the cement mixture may be predicted in operation S130 based on the reactivity calculated in operation S120.

The operation S130 of predicting the products may be performed by a Gibbs free energy minimization method. For example, the operation S130 of predicting the products may be performed by using the following Equation (2).

ΔᵣG°=Σⱼ(ζⱼΔⱼG°ⱼ)=−RT1nK [EQUATION (2)]

In Equation (2), ΔᵣG° represents a change in the Gibbs free energy of the products, R represents a gas constant with a value of 8.3145 Jmol⁻¹K⁻¹, T represents a temperature K, K represents an equilibrium constant, and ζⱼ and ΔⱼG°ⱼ represent constants.

In some embodiments, the products may include components included in concrete. That is, predicting the products in operation S130 may include predicting the components of concrete manufactured by using the cement mixture.

In some embodiments, the products may include portlandite, monosulfate, ettringite, and/or an unreacted admixture. In some embodiments, predicting the products may include predicting volumes of portlandite, monosulfate, ettringite, and/or the unreacted admixture.

In some embodiments, predicting the products in operation S130 may include predicting the products in accordance with the weight ratio wt % of the admixture to a gross weight of the cement mixture. In other words, predicting the products may include predicting changes in volumes of the products in accordance with a change in the weight ratio wt % of the admixture.

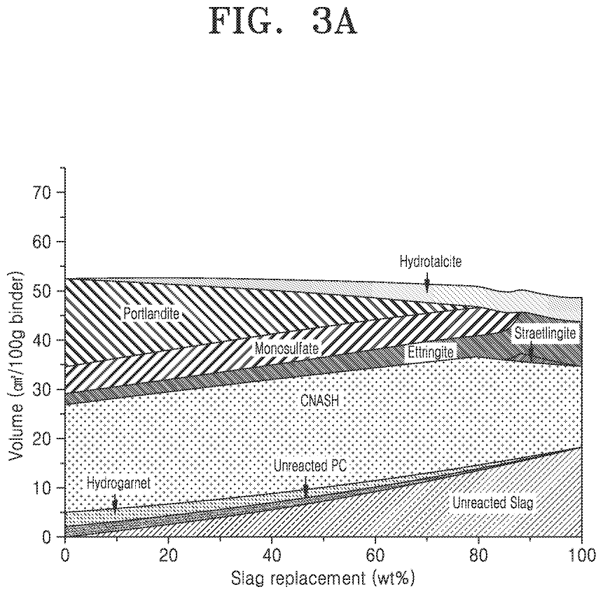

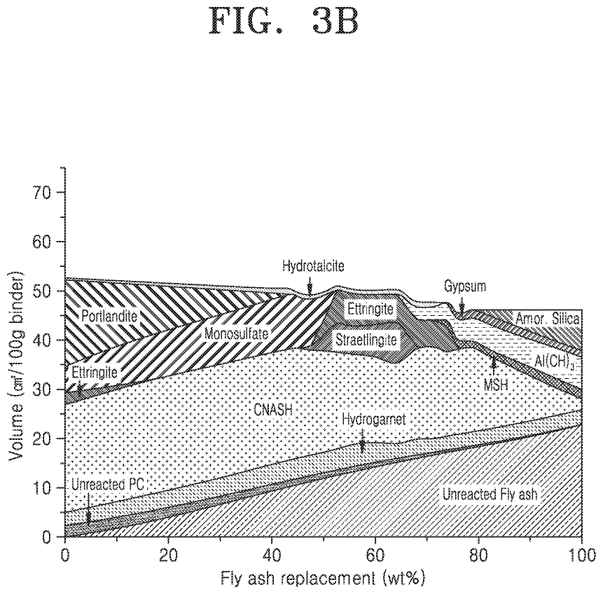

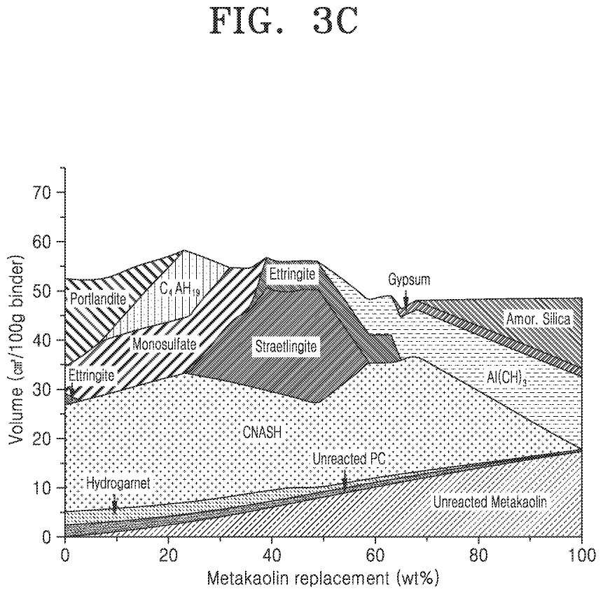

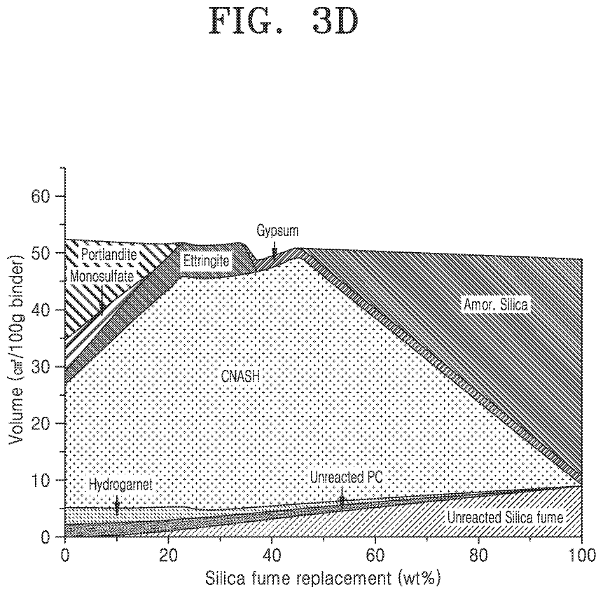

A result of predicting the products of each of the cement mixtures in each of the experimental examples of embodiments in operation S130 is illustrated in each of FIGS. 3A to 3D.

FIGS. 3A to 3D are graphs each illustrating the result of predicting the products of each of the cement mixtures in operation S130 by the method S100 of predicting the concrete characteristics according to embodiments. In each of the graphs of FIGS. 3A to 3D, the vertical axis represents volume, and the unit of volume is cm³/100 g.

Specifically, FIG. 3A is a graph illustrating the result of predicting the products of the cement mixture including slag by the method S100 of predicting the concrete characteristics according to embodiments. Referring to FIG. 3A, it may be predicted that the volumes of the products change as a weight ratio wt % of slag in the cement mixture changes from 0 wt % to 100 wt %.

For example, it may be predicted that the volume of unreacted slag increases as the weight ratio wt % of slag increases in the cement mixture. For example, it may be predicted that the volume of portlandite is reduced as the weight ratio wt % of slag increases, that is, the volume of portlandite tends to change in inverse proportion to the weight ratio wt % of slag. For example, it may be predicted that the volumes of monosulfate, ettringite, and CNASH do not remarkably change until the weight ratio wt % of slag increases from 0 wt % to 80 wt %. In the current specification, CNASH as a material similar to calcium silicate hydrate (CSH) may be amorphous hydrate consisting of CaO—Na₂O—SiO₂—Al₂O₃—H₂O. Hydrate consisting of CaO—Na₂O—SiO₂—Al₂O₃—H₂O used in the present specification represents a material consisting of CaO, Na₂O, SiO₂, Al₂O₃, and H₂O, and does not indicate a stoichiometric relationship thereof.

Specifically, FIG. 3B is a graph illustrating the result of predicting the products of the cement mixture including fly ash by the method S100 of predicting the concrete characteristics according to embodiments. Referring to FIG. 3B, it may be predicted that the volumes of the products change as a weight ratio wt % of fly ash in the cement mixture changes from 0 wt % to 100 wt %.

For example, because a consumption amount of portlandite in the cement mixture including fly ash is greater than that in the cement mixture including slag, which may be predicted through FIG. 3, it may be predicted that the weight ratio wt % of the admixture in which portlandite does not exist anymore is lower as the weight ratio wt % of the admixture increases.

That is, while it may be predicted that portlandite is produced until the weight ratio wt % of the admixture is about 80 wt % or less in the cement mixture including slag, it may be predicted that portlandite is produced only until the weight ratio wt % of the admixture is about 45 wt % or less in the cement mixture including fly ash. For example, while ettringite is rarely produced in the cement mixture in which the weight ratio wt % of fly ash is about 50 wt % or less, it may be predicted that straetlingite and ettringite start to be produced when the weight ratio wt % of fly ash increases.

Specifically, FIG. 3C is a graph illustrating the result of predicting the products of the cement mixture including metakaolin by the method S100 of predicting the concrete characteristics according to embodiments. Referring to FIG. 3C, it may be predicted that the volumes of the products change as a weight ratio wt % of metakaolin in the cement mixture changes from 0 wt % to 100 wt %.

For example, it may be predicted that production of portlandite is reduced and production of monosulfate and C₄AH₁₉ increases at the beginning as the weight ratio wt % of metakaolin increases in the cement mixture. For example, it may be predicted that straetlingite and ettringite are produced in the cement mixture in which the weight ratio wt % of metakaolin is greater than 35 wt %. For example, when the weight ratio wt % of metakaolin is 50 wt % or more, it may be predicted that Al(OH)₃ and/or amorphous silica hydrate start to be produced, which may be predicted as being caused by excessive incorporation of SiO₂.

For example, the changes in volumes of the products in the cement mixture including metakaolin are similar to the changes in volumes of the products in the cement mixture including slag and/or fly ash, which may be predicted through FIGS. 3A and 3B.

Specifically, FIG. 3D is a graph illustrating the result of predicting the products of the cement mixture including silica fume by the method S100 of predicting the concrete characteristics according to embodiments. Referring to FIG. 3D, it may be predicted that the volumes of the products change as a weight ratio wt % of silica fume in the cement mixture changes from 0 wt % to 100 wt %.

For example, it may be predicted that the weight ratio wt % of the admixture in which portlandite is not produced in the cement mixture including silica fume is similar to the weight ratio wt % of the admixture in which portlandite is not produced in the cement mixture including metakaolin, which may be predicted through FIG. 3C. For example, while reduction in portlandite in the cement mixture including metakaolin causes production of CNASH, it may be predicted that reduction in portlandite in the cement mixture including silica fume mainly causes production of monosulfate. For example, when the weight ratio wt % of silica fume in the cement mixture is greater than about 50 wt %, it may be predicted that amorphous silica starts to be produced. In some embodiments, because amorphous silica has a low contribution to strength of concrete including the same, it may be predicted that a proper weight ratio wt % of silica fume in the cement mixture is less than about 45 wt %.

With reference to operation S130 and each of FIGS. 3A to 3D, it is possible to predict the products in accordance with the change in the weight ratio wt % of the admixture in each of the cement mixtures including slag and other admixtures.

Referring to FIG. 1, the concrete characteristics may be predicted in operation S140 based on the products predicted in operation S130. Concrete may include the products predicted through operation S130 in accordance with the weight ratio wt % of the admixture.

In some embodiments, predicting the concrete characteristics in operation S140 may include predicting the amount of an air gap in concrete in operation S141 based on the predicted products. The amount of an air gap in concrete may be predicted in operation S141 by using the following Equation (3).

an amount of air gap %=(1−a volume of a final result/a volume at the beginning)*100% [EQUATION (3)]

In some embodiments, predicting the amount of an air gap in concrete in operation S141 may include predicting the amount of an air gap in concrete in accordance with the weight ratio wt % of the admixture to the gross weight of the cement mixture. In other words, predicting the amount of an air gap in concrete may include predicting a change in the amount of an air gap in concrete in accordance with the change in the weight ratio wt % of the admixture.

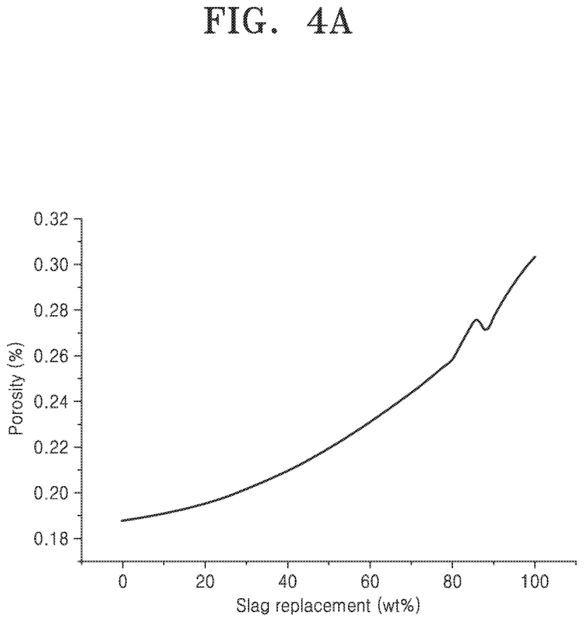

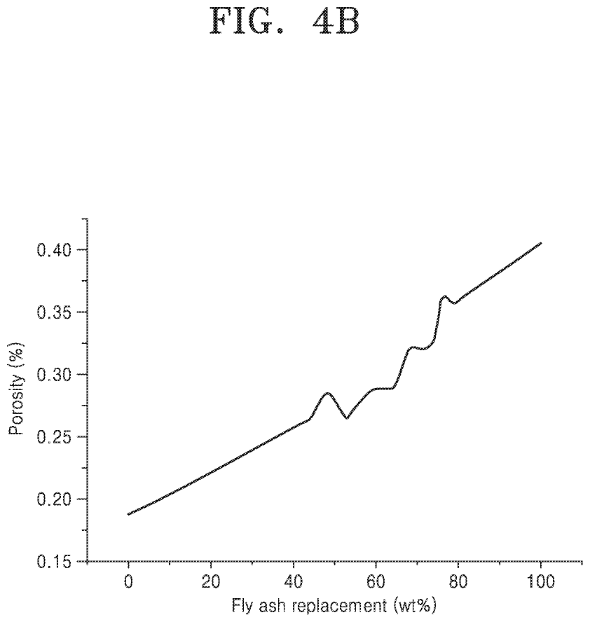

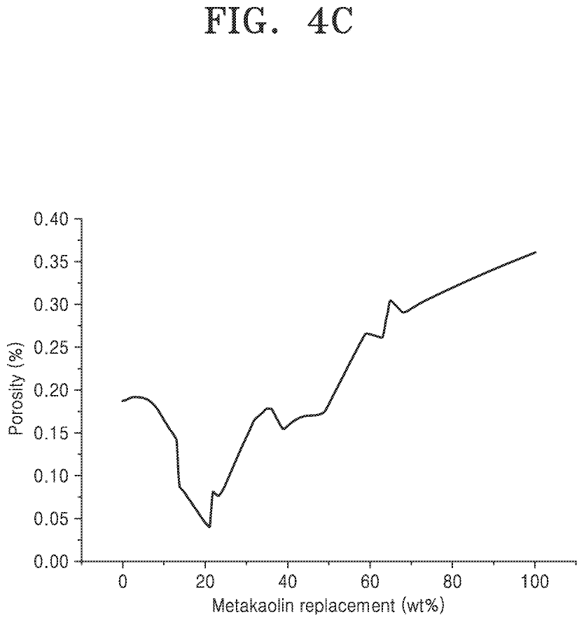

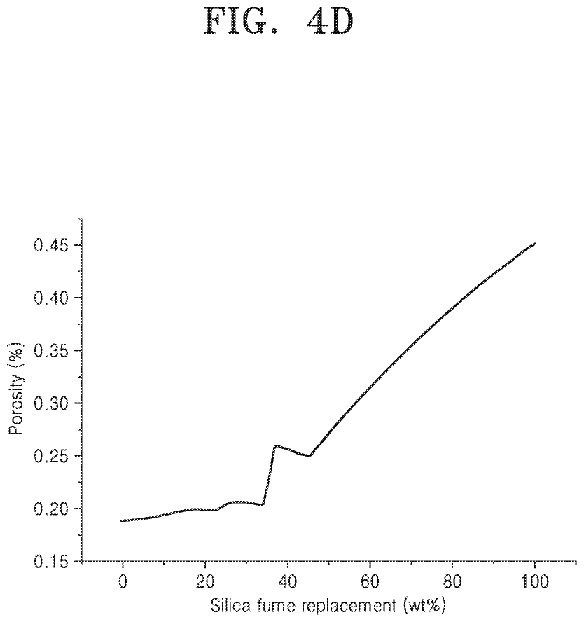

A result of predicting the amount of an air gap in concrete in each of the experimental examples of embodiments in operation S141 is illustrated in each of FIGS. 4A to 4D.

FIGS. 4A to 4D are graphs each illustrating the result of predicting the amount of an air gap in concrete by the method S100 of predicting the concrete characteristics according to embodiments. In each of the graphs of FIGS. 4A to 4D, the vertical axis represents the amount of the air gap, and the unit of the amount of the air gap is percent %.

Specifically, FIG. 4A is a graph illustrating the result of predicting the amount of an air gap in concrete including slag by the method S100 of predicting the concrete characteristics according to embodiments. Referring to FIG. 4A, the amount of an air gap in concrete produced as a weight ratio wt % of slag in the cement mixture changes from 0 wt % to 100 wt % may be predicted. For example, except for a section in which the weight ratio wt % of slag in the cement mixture is about 85 wt % to about 90 wt %, it may be predicted that the amount of an air gap in concrete increases as the weight ratio wt % of the admixture increases.

Specifically, FIG. 4B is a graph illustrating the result of predicting the amount of an air gap in concrete including fly ash by the method S100 of predicting the concrete characteristics according to embodiments. Referring to FIG. 4B, the amount of an air gap in concrete produced as a weight ratio wt % of fly ash in the cement mixture changes from 0 wt % to 100 wt % may be predicted. For example, except for a section in which the weight ratio wt % of fly ash in the cement mixture is about 45 wt % to about 55 wt % and a section in which the weight ratio wt % of fly ash in the cement mixture is about 75 wt % to about 80 wt %, it may be predicted that the amount of an air gap in concrete increases as the weight ratio wt % of the admixture increases.

Specifically, FIG. 4C is a graph illustrating the result of predicting the amount of an air gap in concrete including metakaolin by the method S100 of predicting the concrete characteristics according to embodiments. Referring to FIG. 4C, the amount of an air gap in concrete produced as a weight ratio wt % of metakaolin in the cement mixture changes from 0 wt % to 100 wt % may be predicted. For example, it may be predicted that the amount of an air gap in concrete is reduced as the weight ratio wt % of metakaolin in the cement mixture increases from 0 wt % to about 20 wt %. For example, in most sections in which the weight ratio wt % of metakaolin in the cement mixture is about 20 wt % or more, it may be predicted that the amount of an air gap in concrete increases as the weight ratio wt % of the admixture increases.

Specifically, FIG. 4D is a graph illustrating the result of predicting the amount of an air gap in concrete including silica fume by the method S100 of predicting the concrete characteristics according to embodiments. Referring to FIG. 4D, the amount of an air gap in concrete produced as a weight ratio wt % of silica fume in the cement mixture changes from 0 wt % to 100 wt % may be predicted. For example, it may be predicted that the amount of an air gap in concrete does not remarkably change until the weight ratio wt % of silica fume in the cement mixture increases from 0 wt % to about 35 wt %. For example, the amount of an air gap in concrete may be predicted because the amount of an air gap in concrete does not remarkably change until the weight ratio wt % of silica fume in the cement mixture increases from 0 wt % to about 35 wt %.

With reference to operation S141 and each of FIGS. 4A to 4D, the effect of the change in the weight ratio wt % of each of slag and other admixtures on the amount of an air gap in concrete may be predicted.

In some embodiments, predicting the concrete characteristics in operation S140 may include predicting the compressive strength of concrete in operation S142 based on the amount of an air gap in concrete predicted in operation S141. The compressive strength of concrete may be predicted in operation S142 by using the following Equation (4).

σ=σ₀λ[(1−2p)^{1.85}×(1−P^{2/3})]^{0.5} [EQUATION (4)]

wherein, σ represents the compressive strength of concrete including the admixture of a kind to be predicted, Go represents the compressive strength of the cement mixture that does not include the admixture in a control group, for example, the cement mixture in which the weight ratio wt % of Portland cement is 100 wt %, and p represents the amount % of air gap in concrete predicted in operation S141.

In some embodiments, predicting the compressive strength of concrete in operation S142 may include predicting the compressive strength of concrete in accordance with the weight ratio wt % of the admixture to the gross weight of the cement mixture. In other words, predicting the compressive strength of concrete in operation S142 may include predicting a change in the compressive strength of concrete in accordance with a change in the weight ratio wt % of the admixture.

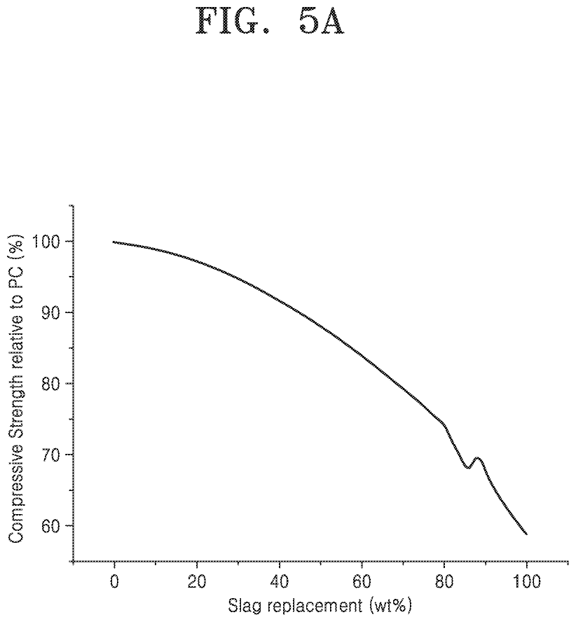

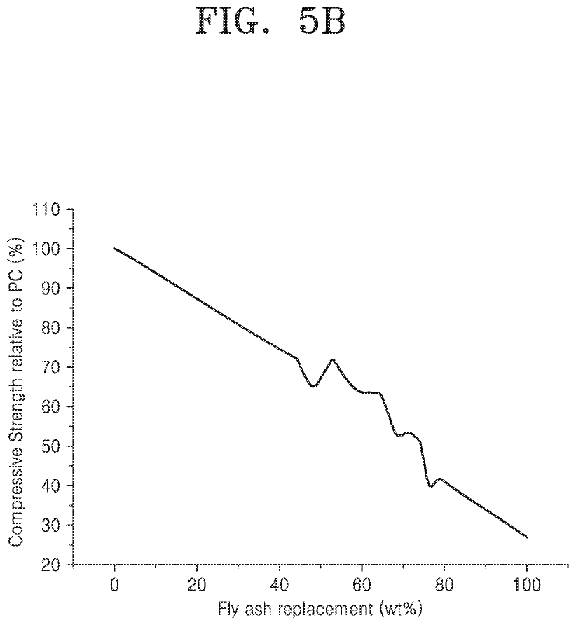

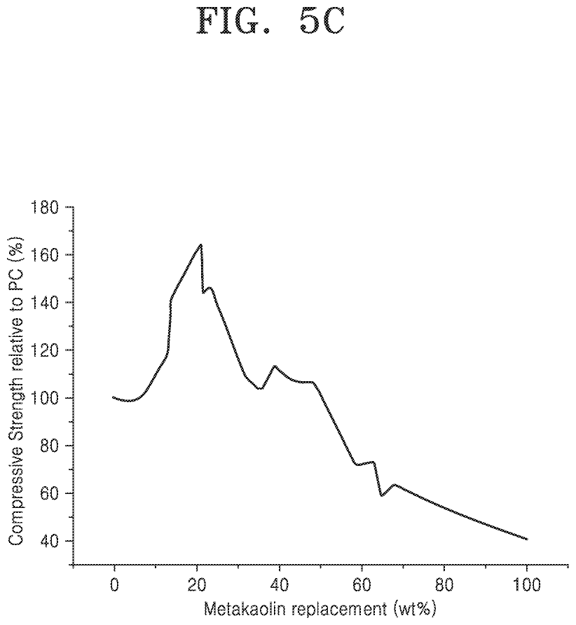

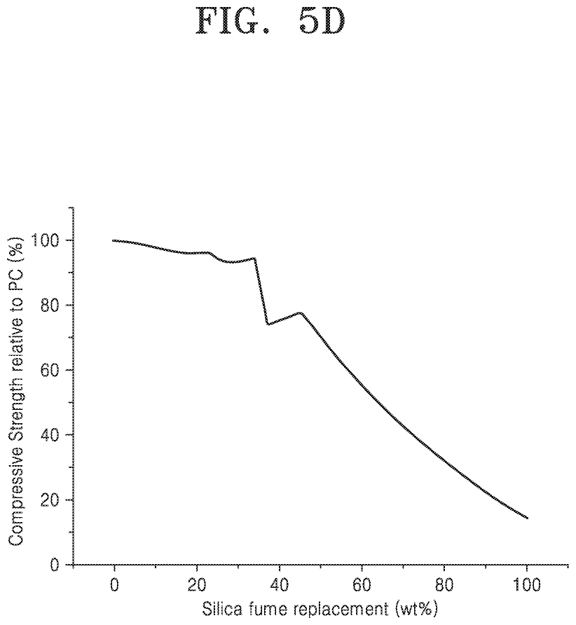

A result of predicting the compressive strength of concrete in each of the experimental examples of embodiments in operation S142 is illustrated in each of FIGS. 5A to 5D.

FIGS. 5A to 5D are graphs each illustrating the result of predicting the compressive strength of concrete by the method S100 of predicting concrete characteristics according to embodiments. In each of the graphs of FIGS. 5A to 5D, the vertical axis represents a ratio % of the compressive strength of each of the cement mixtures compared to the cement mixture that does not include the admixture.

Specifically, FIG. 5A is a graph illustrating the result of predicting the compressive strength of concrete including slag by the method S100 of predicting the concrete characteristics according to embodiments. Referring to FIG. 5A, the compressive strength of concrete produced as a weight ratio wt % of slag in the cement mixture changes from 0 wt % to 100 wt % may be predicted. For example, except for a section in which the weight ratio wt % of slag in the cement mixture is about 85 wt % to about 90 wt %, it may be predicted that the compressive strength of concrete is reduced as the weight ratio wt % of the admixture increases.

Specifically, FIG. 5B is a graph illustrating the result of predicting the compressive strength of concrete including fly ash by the method S100 of predicting the concrete characteristics according to embodiments. Referring to FIG. 5B, the compressive strength of concrete produced as a weight ratio wt % of fly ash in the cement mixture changes from 0 wt % to 100 wt % may be predicted. For example, except for a section in which the weight ratio wt % of fly ash in the cement mixture is about 45 wt % to about 55 wt % and a section in which the weight ratio wt % of fly ash in the cement mixture is about 75 wt % to about 80 wt %, it may be predicted that the compressive strength of concrete is reduced as the weight ratio wt % of the admixture increases.

Specifically, FIG. 5C is a graph illustrating the result of predicting the compressive strength of concrete including metakaolin by the method S100 of predicting the concrete characteristics according to embodiments. Referring to FIG. 5C, the compressive strength of concrete produced as a weight ratio wt % of metakaolin in the cement mixture changes from 0 wt % to 100 wt % may be predicted. For example, it may be predicted that the compressive strength of concrete increases as the weight ratio wt % of metakaolin in the cement mixture increases from 0 wt % to about 20 wt %. For example, in most sections in which the weight ratio wt % of metakaolin in the cement mixture is about 20 wt % or more, it may be predicted that the compressive strength of concrete is reduced as the weight ratio wt % of the admixture increases.

Specifically, FIG. 5D is a graph illustrating the result of predicting the compressive strength of concrete including silica fume by the method S100 of predicting the concrete characteristics according to embodiments. Referring to FIG. 5D, the compressive strength of concrete produced as a weight ratio wt % of silica fume in the cement mixture changes from 0 wt % to 100 wt % may be predicted. For example, it may be predicted that the compressive strength of concrete does not remarkably change until the weight ratio wt % of silica fume in the cement mixture increases from 0 wt % to about 35 wt %. For example, in a section in which the weight ratio wt % of silica fume in the cement mixture is about 50 wt % or more, it may be predicted that the compressive strength of concrete is reduced as the weight ratio wt % of the admixture increases.

With reference to operation S142 and each of FIGS. 5A to 5D of the current specification, the effect of each of slag and other admixtures on the compressive strength of concrete and a change in the compressive strength of concrete may be predicted.

With reference to FIGS. 1 and each of 3A to 5D of the present specification, in the cement mixture including cement and the admixture, it is possible to predict the reactivity of the admixture in accordance with the weight ratio wt % of the admixture, the products, and the amount of an air gap and compressive strength of concrete produced by the cement mixture. That is, with reference to FIGS. 1 and each of 3A to 5D, it is possible to provide a method of predicting the concrete characteristics in accordance with the chemical composition and weight ratio wt % of the admixture.

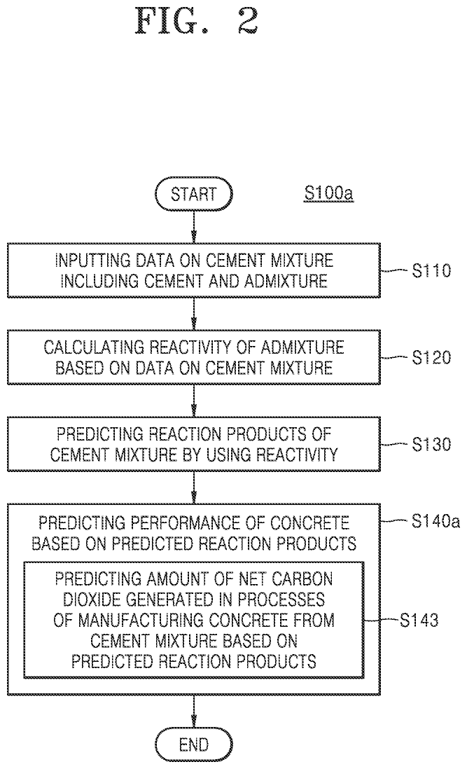

FIG. 2 is a flowchart illustrating a method S100a of predicting concrete characteristics according to embodiments.

Referring to FIG. 2, the concrete characteristics may be predicted in operation S140a based on the products predicted in operation S130. In some embodiments, predicting the concrete characteristics in operation S140a may include predicting an amount of net carbon dioxide generated in processes of manufacturing concrete from the cement mixture based on the predicted products in operation S143.

In some embodiments, predicting the amount of net carbon dioxide in operation S143 may include predicting the amount of net carbon dioxide in accordance with the weight ratio wt % of the admixture to the gross weight of the cement mixture. In other words, predicting the amount of net carbon dioxide may include predicting a change in the amount of net carbon dioxide in accordance with the change in the weight ratio wt % of the admixture.

In some embodiments, predicting the amount of net carbon dioxide in operation S143 may mean excluding a total amount of carbon dioxide absorbed in the processes of manufacturing concrete from the cement mixture from a total amount of carbon dioxide generated in the manufacturing processes. Specifically, the total amount of carbon dioxide absorbed in the manufacturing processes may be obtained by calculating an amount of a reaction product including carbon dioxide, for example, calcium carbonate (CaCO₃), among the products predicted in operation S130.

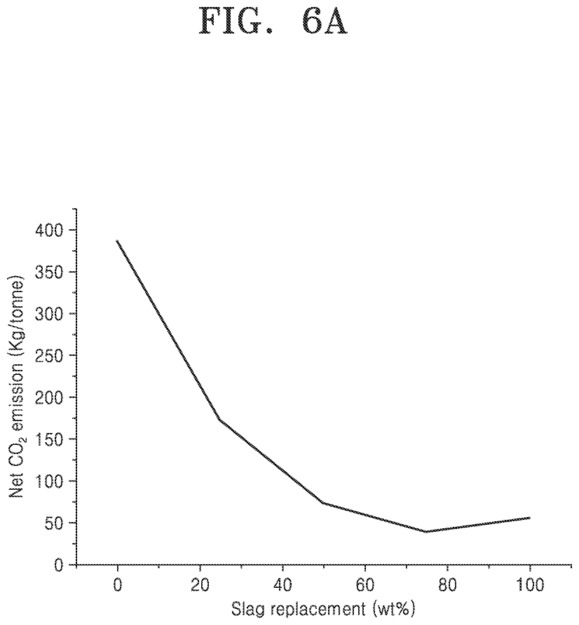

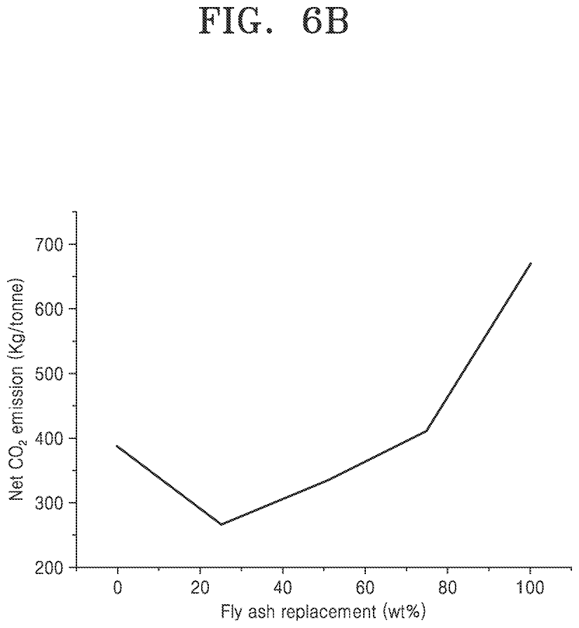

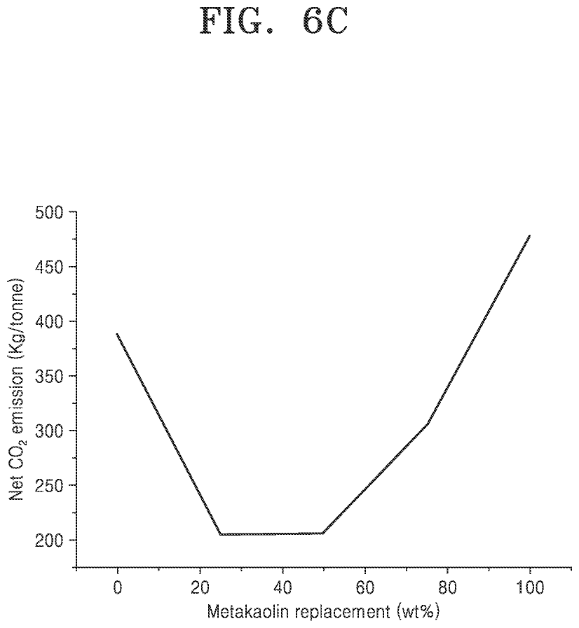

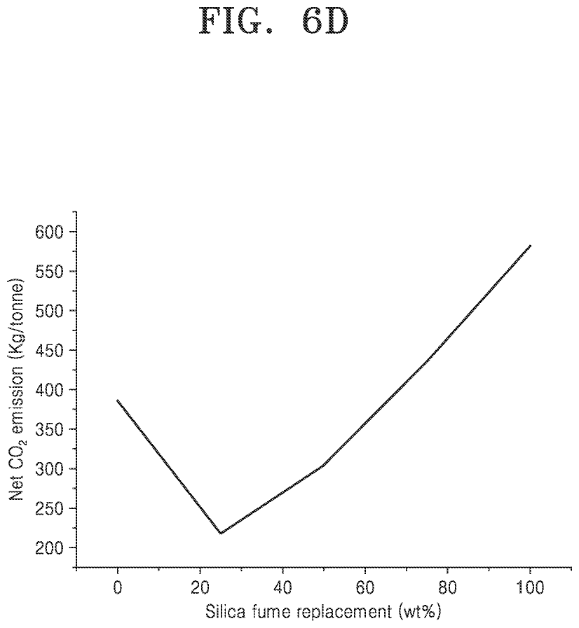

A result of predicting the amount of net carbon dioxide generated in the processes of manufacturing concrete from the cement mixture in each of the experimental examples of embodiments in accordance with operation S143 is illustrated in each of FIGS. 6A to 6D.

FIGS. 6A to 6D are graphs each illustrating the result of predicting the amount of net carbon dioxide generated in the manufacturing processes of concrete by the method of predicting the concrete characteristics according to embodiments. In each of the graphs of FIGS. 6A to 6D, the vertical axis represents the amount of net carbon dioxide, and the unit is Kg/tonne.

Specifically, FIG. 6A is a graph illustrating the result of predicting the amount of net carbon dioxide generated in the manufacturing processes of concrete including slag by the method of predicting the concrete characteristics according to embodiments. Referring to FIG. 6A, it may be predicted that the amount of net carbon dioxide changes as a weight ratio wt % of slag in the cement mixture changes from 0 wt % to 100 wt %.

For example, it may be predicted that the amount of net carbon dioxide is reduced until the weight ratio wt % of slag in the cement mixture increases from 0 wt % to about 75 wt %, because CaO included in slag in a high ratio (about 40 wt % or more in the embodiment) is effective in precipitating carbon dioxide in the form of CaCO₃.

Specifically, FIG. 6B is a graph illustrating the result of predicting the amount of net carbon dioxide generated in the manufacturing processes of concrete including fly ash by the method of predicting the concrete characteristics according to embodiments. Referring to FIG. 6B, it may be predicted that the amount of net carbon dioxide changes as a weight ratio wt % of fly ash in the cement mixture changes from 0 wt % to 100 wt %.

For example, the amount of net carbon dioxide is reduced until the weight ratio wt % of fly ash in the cement mixture increases from 0 wt % to about 22 wt %, so that it may be predicted that the smallest amount of net carbon dioxide is generated in a cement mixture in which the weight ratio wt % of the admixture is about 22 wt %. For example, when the weight ratio wt % of fly ash in the cement mixture is greater than about 50 wt %, it may be predicted that a greater amount of net carbon dioxide is generated than when only cement is used without using the admixture.

Specifically, FIG. 6C is a graph illustrating the result of predicting the amount of net carbon dioxide generated in the manufacturing processes of concrete including metakaolin by the method of predicting the concrete characteristics according to embodiments. Referring to FIG. 6C, it may be predicted that the amount of net carbon dioxide changes as a weight ratio wt % of metakaolin in the cement mixture changes from 0 wt % to 100 wt %.

For example, it may be predicted that the smallest amount of net carbon dioxide is generated in a section in which the weight ratio wt % of metakaolin is about 20 wt % to about 50 wt % in the cement mixture.

Specifically, FIG. 6D is a graph illustrating the result of predicting the amount of net carbon dioxide generated in the manufacturing processes of concrete including silica fume by the method of predicting the concrete characteristics according to embodiments. Referring to FIG. 6D, it may be predicted that the amount of net carbon dioxide changes as a weight ratio wt % of silica fume in the cement mixture changes from 0 wt % to 100 wt %.

For example, the amount of net carbon dioxide is reduced until the weight ratio wt % of silica fume in the cement mixture increases from 0 wt % to about 22 wt %, so that it may be predicted that the smallest amount of net carbon dioxide is generated in a cement mixture in which the weight ratio wt % of the admixture is about 22 wt %, which may be predicted to be similar to the case in which fly ash is used as the admixture, which may be predicted with reference to FIG. 6B.

With reference to FIGS. 2 and each of 6A to 6D of the present specification, it is possible to predict the amount of net carbon dioxide generated in the processes of manufacturing concrete in accordance with the weight ratio wt % of the admixture in the cement mixture including cement and the admixture. That is, with reference to FIGS. 2 and each of 6A to 6D, it is possible to provide a method of predicting the concrete characteristics in accordance with the chemical composition and weight ratio wt % of the admixture.

While aspects of embodiment have been particularly shown and described, it will be understood that various changes in form and details may be made therein without departing from the spirit and scope of the following claims.

What is claimed is:

1. A method of predicting characteristics of concrete, the method comprising:
inputting data of a cement mixture including cement and an admixture;
calculating reactivity of the admixture based on the data; and
predicting products of the cement mixture based on the reactivity.

2. The method of claim 1, wherein the cement comprises Portland cement, and
wherein the admixture comprises at least one of slag, fly ash, metakaolin, and silica fume.

3. The method of claim 1, wherein the data comprises at least one of a mixing ratio of the cement mixture, chemical compositions of the cement and the admixture, a specific surface area of the admixture, a curing condition of the concrete, and a curing time of the concrete.

4. The method of claim 1, wherein, in the calculating of the reactivity of the admixture based on the data, the reactivity is calculated by equation (1):
reactivity(wt %) =47.19×A−0.01144×G+0.1144×I−0.6373×M+0.00185×J ×(M+D×H+H×K)+0.6373×\sqrt{C}+13.16×\sqrt{V}M+E²×(G−I) ×(4×G+H)×0.0001544+0.01144×B×D×K×(F+G+L)−9.762 [EQUATION (1)]
wherein A represents a ratio of a weight of water to a weight of the admixture, B represents a curing temperature ° C., C represents curing time in days, D represents a weight ratio wt % of SiO₂ included in the cement to a gross weight of the cement, E represents a weight ratio wt % of CaO included in the cement to the gross weight of the cement, F represents a weight ratio wt % of MgO included in the cement to the gross weight of the cement, G represents a weight ratio wt % of SO₃ included in the cement to the gross weight of the cement, H represents a weight ratio wt % of Al₂O₃ included in the admixture to the gross weight of the admixture, I represents a weight ratio wt % of Fe₂O₃ included in the admixture to the gross weight of the admixture, J represents a weight ratio wt % of CaO included in the admixture to the gross weight of the admixture, K represents a weight ratio wt % of SO₃ included in the admixture to the gross weight of the admixture, L represents a weight ratio wt % of Na₂O included in the admixture to the gross weight of the admixture, and M represents a specific surface area of the admixture in m²/gm.

5. The method of claim 1, wherein, in the predicting of the products of the cement mixture based on the reactivity, the products are predicted by a Gibbs free energy minimization method calculated by equation (2):
ΔᵣG°=Σⱼ(ζⱼΔⱼG°ⱼ)=−RT1nK [EQUATION (2)]
wherein ΔᵣG° represents a change in Gibbs free energy of the products, R represents a gas constant with a value of 8.3145 Jmol⁻¹K⁻¹, T represents a temperature K, K represents an equilibrium constant, and ζⱼ and ΔⱼG°ⱼ represent constants.

6. The method of claim 1, wherein, in the predicting of the products of the cement mixture based on the reactivity, a volume of at least one of portlandite, monosulfate, ettringite, and an unreacted admixture is predicted.

7. The method of claim 1, wherein, in the predicting of the products of the cement mixture based on the reactivity, the products in accordance with a weight ratio wt % of the admixture to a gross weight of the cement mixture are predicted.

8. The method of claim 1, further comprising predicting characteristics of concrete based on the predicting of the products of the cement mixture based on the reactivity.

9. A method of predicting characteristics of concrete, the method comprising:
inputting data on a cement mixture including cement and an admixture;
calculating reactivity of the admixture based on the data;
predicting products of the cement mixture based on the reactivity; and
predicting characteristics of concrete including the products based on the predicting of the products of the cement mixture based on the reactivity, wherein the predicting of the concrete characteristics comprises predicting an amount of net carbon dioxide generated in processes of manufacturing concrete from the cement mixture,
wherein the amount of net carbon dioxide means excluding an amount of carbon dioxide absorbed in the manufacturing processes from an amount of carbon dioxide generated in the manufacturing processes.

10. The method of claim 9, wherein the predicting of the concrete characteristics comprises predicting an amount of an air gap in the concrete by equation (3) based on the predicting of the products of the cement mixture based on the reactivity:
an amount of air gap %=(1−a volume of a final result/a volume at the beginning)*100% [EQUATION (3)]

11. The method of claim 10, wherein, in the predicting of the amount of the air gap in the concrete, the amount of the air gap in the concrete in accordance with a weight ratio wt % of the admixture to a gross weight of the cement mixture is predicted.

12. The method of claim 9, wherein the predicting of the concrete characteristics comprises:
predicting the amount of an air gap in the concrete based on the predicting of the products of the cement mixture based on the reactivity; and
predicting a compressive strength of concrete by equation (4) based on the predicting of the amount of the air gap in the concrete:
σ=σ₀λ[(1−2p)^{1.85}×(1−P^{2/3})]^{0.5} [EQUATION (4)]
wherein σ represents a compressive strength of concrete including an admixture to be predicted, σ₀ represents a compressive strength of a cement mixture that does not include the admixture, and p represents the predicted amount % of the air gap in the concrete.

13. The method of claim 12, wherein, in the predicting of the compressive strength of the concrete, the compressive strength of the concrete in accordance with a weight ratio wt % of the admixture to a gross weight of the cement mixture is predicted.

14. The method of claim 9, wherein the cement comprises Portland cement, and
wherein the admixture comprises at least one of slag, fly ash, metakaolin, and silica fume.

15. The method of claim 9, wherein, in the predicting of the amount of net carbon dioxide, the amount of net carbon dioxide in accordance with a weight ratio wt % of the admixture to a gross weight of the cement mixture is predicted.

16. A method of predicting concrete characteristics, the method comprising:
inputting data on a cement mixture including cement and an admixture;
calculating reactivity of the admixture based on the data;
predicting products of the cement mixture based on the reactivity;
predicting an amount of an air gap in concrete including the products based on the predicting of the products of the cement mixture based on the reactivity; and
predicting a compressive strength of concrete based on the predicting of the amount of the air gap in the concrete.

17. The method of claim 16, further comprising predicting an amount of net carbon dioxide generated in processes of manufacturing the concrete from the cement mixture based on the predicting of the products of the cement mixture based on the reactivity, wherein the amount of net carbon dioxide means excluding an amount of carbon dioxide absorbed in the manufacturing processes from an amount of carbon dioxide generated in the manufacturing processes.

18. The method of claim 16, wherein the cement comprises Portland cement, and
wherein the admixture comprises at least one of slag, fly ash, metakaolin, and silica fume.

19. The method of claim 16, wherein, in the calculating of the reactivity of the admixture based on the data, the reactivity is calculated by equation (1):
reactivity(wt %) =47.19×A−0.01144×G+0.1144×I−0.6373×M+0.00185×J ×(M+D×H+H×K)+0.6373×\sqrt{C}+13.16×\sqrt{V}M+E²×(G−I) ×(4×G+H)×0.0001544+0.01144×B×D×K×(F+G+L)−9.762 [EQUATION (1)]
wherein A represents a ratio of a weight of water to a weight of the admixture, B represents a curing temperature ° C., C represents curing time in days, D represents a weight ratio wt % of SiO₂ included in the cement to a gross weight of the cement, E represents a weight ratio wt % of CaO included in the cement to the gross weight of the cement, F represents a weight ratio wt % of MgO included in the cement to the gross weight of the cement, G represents a weight ratio wt % of SO₃ included in the cement to the gross weight of the cement, H represents a weight ratio wt % of Al₂O₃ included in the admixture to the gross weight of the admixture, I represents a weight ratio wt % of Fe₂O₃ included in the admixture to the gross weight of the admixture, J represents a weight ratio wt % of CaO included in the admixture to the gross weight of the admixture, K represents a weight ratio wt % of SO₃ included in the admixture to the gross weight of the admixture, L represents a weight ratio wt % of Na₂O included in the admixture to the gross weight of the admixture, and M represents a specific surface area of the admixture in m²/gm.

20. The method of claim 16, wherein, in the predicting of the products of the cement mixture based on the reactivity, the products in accordance with a weight ratio wt % of the admixture to a gross weight of the cement mixture are predicted.
